(19) United States
(12) Patent Application Publication
(10) Pub. No.: US 2024/0238221 A1
(43) Pub. Date: Jul. 18, 2024

(54) TAPENTADOL FOR TREATING NEUROPATHIC PAIN IN COVID-19 PATIENTS

(71) Applicant: Grünenthal GmbH, Aachen (DE)

(72) Inventors: Katharina WENGE, Aachen (DE); Christian ELLING, Aachen (DE)

(73) Assignee: Grünenthal GmbH, Aachen (DE)

(21) Appl. No.: 18/289,685

(22) PCT Filed: May 6, 2022
(86) PCT No.: PCT/EP2022/062266

§ 371 Date: Nov. 6, 2023

(30) Foreign Application Priority Data
May 7, 2021 (EP) 21172686.4

(51) Int. Cl. A61K 31/137; A61K 31/427; A61K 31/4706; A61K 31/513; A61K 31/573; A61K 31/706

(52) U.S. Cl. CPC A61K 31/137; A61K 31/427; A61K 31/4706; A61K 31/513; A61K 31/573; A61K 31/706

(57) ABSTRACT

The invention relates to a pharmaceutical dosage form comprising Tapentadol or a physiologically acceptable salt thereof for use in the treatment of pain in a patient who is or who was previously infected with a coronavirus.

Priority is claimed of European patent application no. 21 172 686.4 that was filed on May 7, 2021.

The invention relates to a pharmaceutical dosage form comprising Tapentadol or a physiologically acceptable salt thereof for use in the treatment of pain, preferably neuropathic pain, in a patient who is or who was previously infected with a coronavirus, preferably severe acute respiratory syndrome coronavirus 2.

A significant number of patients currently undergoing or previously having undergone coronavirus disease 2019 (COVID-19) require pain treatment. The painful condition may already have preexisted prior to infection with severe acute respiratory syndrome coronavirus 2 (SARS CoV-2) and may have worsened in the course of COVID-19, or the painful condition may have developed in the course and/or as a consequence of COVID-19. Further, the painful condition may be acute or chronic, typically neuropathic, i.e. caused by damage or disease affecting the somatosensory nervous system.

Various painful conditions can be associated to COVID-19 disease. For example, F. Aksan et al., Journal of NeuroVirology, https://doi.org/10.1007/s13365-020-00887-4 relates to a COVID-19 patient with intense burning pain. A. A. Joyce et al., Pain Medicine, 00(0), 2020, 1-12 relates to changes in interventional pain physician decision-making, practice patterns, and mental health during the early phase of the SARS-CoV-2 global pandemic. H. I. Kemp et al., British Journal of Anaesthesia, 125 (4): 436e449 (2020) relates to implications for rehabilitation with regard to chronic pain after COVID-19. L. Killion et al., BMJ Case Rep 2021; 14:e240863. doi:10.1136/bcr-2020-240863 relates to rare cutaneous manifestation of COVID-19. Ch. H. Meyer-Frießem et al., www.painreportsonline.com, 6 (2021) e893, 1-7, http://dx.doi.org/10.1097/PR9.0000000000000893 is a narrative review of current knowledge of pain during and after COVID-19 in Germany and worldwide. A. Nalbandian et al., Nature Medicine, 27, pages601-615(2021), https://doi.org/10.1038/s41591-021-01283-z relates to post-acute COVID-19 syndrome. J. Blanch-Rubió et al., AGING 2020, Vol. 12, No. 20, relates to the influence of anti-osteoporosis treatments on the incidence of COVID-19 in patients with non-inflammatory rheumatic conditions. W. Sun et al., Frontiers in Microbiology, 24 Sep. 2020 | https://doi.org/10.3389/fmicb.2020.572318 relates to the management of immunity alteration-induced chronic pain during the coronavirus disease-2019 (COVID-19) pandemic. I. P. E. Widyadharma et al., The Egyptian Journal of Neurology, Psychiatry and Neurosurgery (2020) 56:121 https://doi.org/10.1186/s41983-020-00258-0 is a literature review with regard to pain as clinical manifestations of COVID-19 infection and its management in the pandemic era.

Among these painful conditions, neuropathies appear to be predominant and various scientific publications pertain to neuronal implications of COVID-19 and COVID-19 associated painful neuropathies. For example, L. Abdelnour et al., Journal of the Formosan Medical Association, Volume 119, Issue 6, June 2020, Pages 1119-1120 relates to COVID 19 infection presenting as motor peripheral neuropathy. A. N. Özdag Acarli et al., Arch Neuropsychiatry 2020; 57:154-159 relates to the observation of neurological findings and symptoms during the combat against a pandemic with regard to coronavirus disease 2019 (COVID-19) from the point of view of neurologists. S. Andalib et al., Current Neurology and Neuroscience Reports (2021) 21: 9, https://doi.org/10.1007/s11910-021-01102-5 relates to peripheral nervous system manifestations associated with COVID-19. N. Attal et al., www.painreportsonline.com, 6 (2021) e884, 1-6, http://dx.doi.org/10.1097/PR9.0000000000000884 relates to the potential for increased prevalence of neuropathic pain after the COVID-19 pandemic. B. L. Bureau et al., (Nov. 12, 2020), Cureus 12(11):e11452. DOI 10.7759/cureus.11452 relates to peripheral neuropathy as a complication of SARS-Cov-2. M. Cacciavillani et al., Muscle & Nerve, DOI: 10.1002/mus.27083, E7/8 relates to pure sensory neuralgic amyotrophy in COVID-19 infection. X. Cao et al., Journal of Pain Research 2020:13 2361-2365 relates to a case report regarding herpes zoster and postherpetic neuralgia in an elderly patient with critical COVID-19. A. J. McFarland et al., www.painreportsonline.com, 6 (2021) e885, 1-10, http://dx.doi.org/10.1097/PR9.0000000000000885 relates to implications for COVID-19 and pain with regard to neurobiology of SARS-CoV-2 interactions with the peripheral nervous system. D. Kersebaum et al., www.painreportsonline.com, 5 (2020) e858 1-10, http://dx.doi.org/10.1097/PR9.0000000 000000858 relates to the early influence of COVID-19 pandemic associated restrictions on pain, mood, and everyday life of patients with painful polyneuropathy. C. Miller et al., Physical Therapy, 2021; 101:1-8 is a retrospective case series of 15 patients in critical care having brachial plexus neuropathies during the COVID-19 pandemic. P. Novak, eNeurologicalSci Volume 21, December 2020, 100276 is a case report with regard to post COVID-19 syndrome associated with orthostatic cerebral hypoperfusion syndrome, small fiber neuropathy and benefit of immunotherapy.

With regard to the pain treatment by means of opioids, the opioid crises in the United States has been described as an opioid epidemic within the COVID-19 pandemic. For example, J. K. Niles et al., POPULATION HEALTH MANAGEMENT, Volume 24, Number S1, 2021, DOI: 10.1089/pop.2020.0230 relates to drug testing in 2020 with regard to the opioid epidemic within the COVID-19 pandemic. Q. Q. Wang et al., Molecular Psychiatry (2021) 26:30-39, https://doi.org/10.1038/s41380-020-00880-7 relates to analyses from electronic health records in the United States with regard to COVID-19 risk and outcomes in patients with substance use disorders.

M. Angulo-Ardoy et al., Family Practice, 2021, i45-i47 discloses that COVID-19 is an emergent infection, the long-term complications of which are still under study. While hypercoagulability is a common feature in severe cases, the incidence of ischemic complications such as osteonecrosis remains unknown. Previous studies on SARS-CoV1 found an increase in osteonecrosis 3-36 months after infection, and it is still unclear if this was related to the use of corticosteroids or to the virus itself. A 78-year-old woman complained of right knee pain and swelling a month after COVID-19 infection onset. Her knee radiography showed no significant changes compared to previous ones. MRI, on the other hand, found osteonecrosis in the internal femoral condyle. No coagulation abnormalities were found in blood tests. While knee replacement should be her main treatment, it was long delayed due to the COVID-19 pandemic. In the meantime, her tapentadol and salicylic acid doses were increased.

S. Natoli et al., Eur Rev Med Pharmacol Sci, 2021, 25: 4854-4867 discusses the available information regarding the currently utilized COVID-19 therapies and opioids for chronic pain with a focus on warnings of potential interactions between these two therapeutic approaches. Tapentadol is associated with a low risk of drug-drug interactions at the CYP450 level and may, therefore, be used in poly-treated patients. Furthermore, Tapentadol exhibits a lower immunosuppressive activity at analgesic doses vs. Morphine. The major elimination pathway for tapentadol is conjugation with glucuronic acid mediated via UGT mainly UGT1A6, UGT1A9 and UGT2B7 isoforms. Thus, concomitant administration with strong inhibitors of these isoenzymes may lead to increased systemic exposure of Tapentadol. Tapentadol is preferred over opioids metabolized through the CYP450 hepatic system.

The known treatment options for pain, preferably neuropathic pain, in patients who are currently infected or who were previously infected with a coronavirus, preferably severe acute respiratory syndrome coronavirus 2, are not satisfactory in every respect and there is a demand for improved treatment options.

It is an object of the invention to provide medicaments for the treatment of pain, preferably neuropathic pain, in patients who are currently infected or who were previously infected with a coronavirus, preferably with severe acute respiratory syndrome coronavirus 2. The medicaments should have advantages. In particular, the medicaments should provide improved pain treatment at higher safety and/or improved tolerability. Further, the medicaments should have neglectable or no interactions with other medicaments which are used for treating other aspects of COVID-19 disease.

This object has been achieved by the subject-matter of the patent claims.

A first aspect of the invention relates to a pharmaceutical dosage form comprising Tapentadol or a physiologically acceptable salt thereof for use in the treatment of pain in a patient who is or who was previously infected with a coronavirus, preferably with SARS CoV-2 virus.

Tapentadol (ATCN02AX06, Palexia®, Nucynta©) is a centrally acting opioid analgesic of the benzenoid class with a dual mode of action as an agonist of the μ-opioid receptor and as a norepinephrine reuptake inhibitor (NRI). Unlike tramadol, it has only weak serotonin reuptake inhibition and is a significantly more potent opioid agonist with no known active metabolites. Tapentadol is not a pro-drug and therefore does not rely on metabolism to produce its therapeutic effects; this provides a consistent dosage-response range among the patient population (https://en.wikipedia.org/wiki/Tapentadol). The analgesic efficacy of tapentadol in the investigated dose ranges is comparable to that of typical strong opioids such as morphine, oxycodone or hydromorphone.

Tapentadol is used for the treatment of moderate to severe pain, specifically in patients with chronic low back pain and pain due to osteoarthritis. S. C. Erosa et al., Current Pain and Headache Reports, (2021) 25:18 is a systematic review of Tapentadol, Buprenorphine, and Levorphanol for the treatment of neuropathic pain.

The medicament according to the invention comprises Tapentadol or a physiologically acceptable salt thereof. Preferred physiologically acceptable salts are selected from the hydrochloride, hydrobromide, dihydrogenphosphate, oxalate, maleate and tartrate.

The patient to be treated according to the invention is or was previously infected with a coronavirus, preferably with SARS CoV-2 virus.

For the purpose of the description, a patient who “is infected” with a coronavirus currently has a detectable viral load above the threshold of suitable means for detecting coronavirus, preferably SARS CoV-2 virus. Suitable means for detecting coronavirus are known to the skilled person and include but are not limited to antibody tests, such as SARS-CoV-2 rapid antibody tests based upon chromatographic immune assays. Other tests are PCR test. Preferably, coronavirus and SARS CoV-2 virus in a patient who “is infected” are determined by means of PCR tests.

For the purpose of the description, a patient who “was infected” with a coronavirus previously had a detectable viral load above the threshold of suitable means for detecting coronavirus, preferably SARS CoV-2 virus, preferably determined by antibody tests (see above). It is contemplated that the patient was never tested during the acute phase and therefore never had a documented positive PCR test. Typically, such patient currently has no detectable viral load above the threshold of suitable means for detecting coronavirus, preferably SARS CoV-2 virus, preferably determined by PCR. Suitable means for determining whether a patient was previously infected with a coronavirus, preferably SARS CoV-2 virus, are known to the skilled person. Preferably, coronavirus and SARS CoV-2 virus in a patient who “was infected” are determined by means of antibody tests. The antibodies are preferably directed towards the nucleocapsid or one or more spike proteins of the coronavirus.

For the purpose of the description, a patient who “is infected” with a coronavirus does not need to show any signs or symptoms of the infection (e.g. COVID-19). Likewise, a patient who “was infected” with a coronavirus does not need to show signs or symptoms of the infection (e.g. COVID-19).

Thus, infection with coronavirus, preferably SARS-CoV-2 virus, means (i) that the patient currently carries a viral load, or (ii) that the patient previously carried a viral load, whereas depending upon the time lapsed from viral infection, said patient typically may have detectable antibodies against coronavirus, preferably SARS-CoV-2 virus, produced due to an immune response to the previous infection.

While the type of coronavirus according to the invention is principally not particularly limited, the coronavirus is preferably a so-called severe acute respiratory syndrome-related coronavirus, more preferably a severe acute respiratory syndrome coronavirus 2 (SARS CoV-2).

In preferred embodiments, the coronavirus is the severe acute respiratory syndrome coronavirus 2 or a mutant of the severe acute respiratory syndrome coronavirus 2 selected from the group consisting of Cluster 5, Lineage B.1.1.7, Lineage B.1.1.207, Lineage B.1.1.317, Lineage B.1.1.318, Lineage B.1.429, Lineage B.1.525, Lineage B.1.526, Lineage B.1.618, Lineage P.1, and Lineage P.3.

Preferably, the patient suffers or suffered from a corona virus disease (COVID), preferably COVID-19.

For the purpose of the description, a patient “suffers” (is suffering) from a corona virus disease (COVID), preferably COVID-19, when the patient currently has signs or symptoms of corona virus disease (COVID), preferably COVID-19, and currently typically has a detectable viral load.

For the purpose of the description, a patient “suffered” from a corona virus disease (COVID), preferably COVID-19, when the patient previously had signs or symptoms of corona virus disease (COVID), preferably COVID-19, but (i) currently—besides the pain to be treated—does not necessarily still show any of such signs or symptoms any longer, or (ii) currently only shows a limited number of such signs or symptoms, or (iii) currently shows such signs or symptoms to a significant lower degree than previously.

Characteristic signs and symptoms of corona virus disease (COVID) and COVID-19 are known to the skilled person and include but are not limited to severe acute respiratory syndrome, cough, fever, cold, disturbance of olfactory sense, disturbance of gustatory sense, pneumonia, sore throat, breathlessness (dyspnea), headache, limb pain, anorexia, weight loss, nausea, vomiting, diarrhea, stomach ache, conjunctivitis, skin rash, sentinel node swelling, apathy, and somnolence.

Preferably, the corona virus disease is a severe acute respiratory syndrome (SARS), preferably SARS-CoV-2. In preferred embodiments, the patient is under intensive care.

In a preferred embodiment, the patient suffers from chronic corona virus disease syndrome (CCS), which is also known as “long COVID”, “post-acute sequelae of SARS-CoV-2 infection”, “post-acute sequelae of COVID-19 (PASC)”, or “long-haul COVID”.

In preferred embodiments, the patient suffers from
(i) acute COVID-19 (signs and symptoms of COVID-19 for up to 4 weeks);
(ii) ongoing symptomatic COVID-19 (signs and symptoms of COVID-19 from 4 to 12 weeks);
(iii) post-COVID-19 syndrome (signs and symptoms that develop during or after an infection consistent with COVID-19, continue for more than 12 weeks and are not explained by an alternative diagnosis); or
(iv) long COVID (signs and symptoms that continue or develop after acute COVID-19; includes both ongoing symptomatic COVID-19 and post-COVID-19 syndrome, i.e. (ii) and (iii) defined above).

For the purpose of the description, the above stages (i) to (iv) of COVID-19 are defined in accordance with the COVID-19 rapid guideline: managing the long-term effects of COVID-19, NICE guideline [NG188], published on 18 Dec. 2020.

For the purpose of the description, “COVID-19 neuropathic pain” refers to neuropathic pain due to SARS-CoV-2 infection, neuropathic pain linked to SARS-CoV-2 infection, COVID-19-associated neuropathy, and the like.

In preferred embodiments, the patient suffers, or suffered or has suffered from a neurological disorder or disease, preferably selected from Guillain-Barré syndrome, myelitis and stroke.

Preferably, the neurological disorder or disease is due to corona virus disease (COVID), preferably COVID-19.

In preferred embodiments, prior to infection with the coronavirus, the patient suffered or has suffered from
(i) pre-existing neurological injury; and/or
(ii) pre-existing neuropathic pain, preferably pre-existing chronic neuropathic pain, more preferably pre-existing chronic polyneuropathic pain or pre-existing chronic mononeuropathic pain.

For the purpose of the description, a patient who “suffered” from (i) pre-existing neurological injury and/or (ii) pre-existing neuropathic pain prior to infection with the coronavirus was diagnosed accordingly before infection with the coronavirus occurred. A patient who “has suffered” from (i) preexisting neurological injury and/or (ii) pre-existing neuropathic pain prior to infection with the coronavirus was likewise diagnosed accordingly before infection with the coronavirus occurred, but currently is still suffering from the (i) pre-existing neurological injury and/or (ii) pre-existing neuropathic pain, i.e. the (i) pre-existing neurological injury and/or (ii) pre-existing neuropathic pain existed already before viral infection and is still currently ongoing or is aggravated by the COVID-19 infection.

Preferably, the pre-existing neuropathic pain is selected from the group consisting of postherpetic neuralgia (PHN), chronic painful radiculopathy, cervical radiculopathy, diabetic painful neuropath (DPN), spinal cord injury pain, neuropathic pain due to myelitis, neuropathic pain due to cancer, trigeminal neuralgia, neuropathic pain associated with Guillain-Barré syndrome, chemotherapy induced neuropathic pain, small-fiber neuropathy, chronic idiopathic axonal polyneuropathy, post-traumatic neuropathic pain, post-surgical neuropathic pain, HIV infection-induced neuropathic pain, and poststroke pain.

In other preferred embodiments, prior to infection with the coronavirus, the patient neither suffered from (i) pre-existing neurological injury nor from (ii) pre-existing neuropathic pain.

Preferably, the pain to be treated with Tapentadol or a physiologically acceptable salt thereof is neuropathic pain, preferably peripheral neuropathic pain. Preferably, the pain to be treated is selected from the group consisting of COVID-19 neuropathic pain (COVID-19-associated neuropathy), postherpetic neuralgia (PHN), chronic painful radiculopathy, cervical radiculopathy, diabetic painful neuropath (DPN), spinal cord injury pain, neuropathic pain due to myelitis, neuropathic pain due to cancer, trigeminal neuralgia, neuropathic pain associated with Guillain-Barré syndrome, chemotherapy induced neuropathic pain, small-fiber neuropathy, chronic idiopathic axonal polyneuropathy, post-traumatic neuropathic pain, post-surgical neuropathic pain, HIV infection-induced neuropathic pain, and poststroke pain.

Preferably, when the patient suffered or has suffered from pre-existing neuropathic pain, preferably pre-existing chronic neuropathic pain, more preferably pre-existing chronic polyneuropathic pain or pre-existing chronic mononeuropathic pain; the pain to be treated is of the same type as the preexisting neuropathic pain.

Preferably, when the patient suffered or has suffered from pre-existing neuropathic condition, preferably neuropathic pain, preferably pre-existing chronic neuropathic pain, more preferably pre-existing chronic polyneuropathic pain or pre-existing chronic mononeuropathic pain; the pre-existing neuropathic condition may still be there, or may be deteriorated, but there are additional symptoms due to COVID-19 itself or due to the treatment received during the acute phase of COVID-19.

The neuropathic pain to be treated according to the invention may cause neuropathic symptoms such as numbness, tingling, pain or impaired sensory function in hands and/or feet, clumsiness in fingers, peripheral muscular weakness, or difficulties in walking. Further symptoms and signs according to the invention may be tingling; numbness; sharp, burning, shooting, or electric-like pain; paresthesia; allodynia; hyperalgesia and hyperpathia; motor dysfunction (for example foot or wrist drop, symmetric motor weakness, difficulty buttoning a shirt or holding a pen); and myalgias or muscle cramps.

Preferably, the neuropathic pain to be treated is associated with sensory loss; preferably hypersensitivity, loss of sensation, or functional deficits such as numbness, tingling, and discomfort in the fingertips and toes, ascending from distal to proximal as the condition progresses.

In preferred embodiments, the pain to be treated is
(i) chronic; and/or
(ii) moderate to severe; and/or
(iii) due to or linked to infection with the coronavirus (e.g. COVID-19 neuropathic pain).

It is contemplated and preferred that treatment with Tapentadol is continued also after the acute phase of the COVID-19 infection has ended.

According to particularly preferred embodiments of the invention, the patient
suffers from acute COVID-19 (NICE guideline [NG188]);
prior to infection with the coronavirus, suffered from (i) pre-existing neurological injury and/or (ii) pre-existing neuropathic pain, preferably pre-existing chronic neuropathic pain, more preferably preexisting chronic polyneuropathic pain or pre-existing chronic mononeuropathic pain;
wherein the pain to be treated is neuropathic pain, preferably peripheral neuropathic pain; more preferably selected from the group consisting of COVID-19 neuropathic pain, postherpetic neuralgia (PHN), chronic painful radiculopathy, cervical radiculopathy, diabetic painful neuropath (DPN), spinal cord injury pain, neuropathic pain due to myelitis, neuropathic pain due to cancer, trigeminal neuralgia, neuropathic pain associated with Guillain-Barré syndrome, chemotherapy induced neuropathic pain, small-fiber neuropathy, chronic idiopathic axonal polyneuropathy, post-traumatic neuropathic pain, post-surgical neuropathic pain, HIV infection-induced neuropathic pain, and poststroke pain.

According to particularly preferred embodiments of the invention, the patient
suffers from acute COVID-19 (NICE guideline [NG188]);
prior to infection with the coronavirus, has suffered from (i) pre-existing neurological injury and/or (ii) pre-existing neuropathic pain, preferably pre-existing chronic neuropathic pain, more preferably pre-existing chronic polyneuropathic pain or pre-existing chronic mononeuropathic pain;
   wherein the pain to be treated is neuropathic pain, preferably peripheral neuropathic pain; more preferably selected from the group consisting of COVID-19 neuropathic pain, postherpetic neuralgia (PHN), chronic painful radiculopathy, cervical radiculopathy, diabetic painful neuropath (DPN), spinal cord injury pain, neuropathic pain due to myelitis, neuropathic pain due to cancer, trigeminal neuralgia, neuropathic pain associated with Guillain-Barré syndrome, chemotherapy induced neuropathic pain, small-fiber neuropathy, chronic idiopathic axonal polyneuropathy, post-traumatic neuropathic pain, post-surgical neuropathic pain, HIV infection-induced neuropathic pain, and poststroke pain.

According to particularly preferred embodiments of the invention, the patient
suffers from acute COVID-19 (NICE guideline [NG188]);
prior to infection with the coronavirus, neither suffered from (i) pre-existing neurological injury nor from (ii) pre-existing neuropathic pain;
   wherein the pain to be treated is neuropathic pain, preferably peripheral neuropathic pain; more preferably COVID-19 neuropathic pain.

According to particularly preferred embodiments of the invention, the patient
suffers from ongoing symptomatic COVID-19 (NICE guideline [NG188]);
prior to infection with the coronavirus, suffered from (i) pre-existing neurological injury and/or (ii) pre-existing neuropathic pain, preferably pre-existing chronic neuropathic pain, more preferably preexisting chronic polyneuropathic pain or pre-existing chronic mononeuropathic pain;
   wherein the pain to be treated is neuropathic pain, preferably peripheral neuropathic pain; more preferably selected from the group consisting of COVID-19 neuropathic pain, postherpetic neuralgia (PHN), chronic painful radiculopathy, cervical radiculopathy, diabetic painful neuropath (DPN), spinal cord injury pain, neuropathic pain due to myelitis, neuropathic pain due to cancer, trigeminal neuralgia, neuropathic pain associated with Guillain-Barré syndrome, chemotherapy induced neuropathic pain, small-fiber neuropathy, chronic idiopathic axonal polyneuropathy, post-traumatic neuropathic pain, post-surgical neuropathic pain, HIV infection-induced neuropathic pain, and poststroke pain.

According to particularly preferred embodiments of the invention, the patient
suffers from ongoing symptomatic COVID-19 (NICE guideline [NG188]);
prior to infection with the coronavirus, has suffered from (i) pre-existing neurological injury and/or (ii) pre-existing neuropathic pain, preferably pre-existing chronic neuropathic pain, more preferably pre-existing chronic polyneuropathic pain or pre-existing chronic mononeuropathic pain;
   wherein the pain to be treated is neuropathic pain, preferably peripheral neuropathic pain; more preferably selected from the group consisting of COVID-19 neuropathic pain, postherpetic neuralgia (PHN), chronic painful radiculopathy, cervical radiculopathy, diabetic painful neuropath (DPN), spinal cord injury pain, neuropathic pain due to myelitis, neuropathic pain due to cancer, trigeminal neuralgia, neuropathic pain associated with Guillain-Barré syndrome, chemotherapy induced neuropathic pain, small-fiber neuropathy, chronic idiopathic axonal polyneuropathy, post-traumatic neuropathic pain, post-surgical neuropathic pain, HIV infection-induced neuropathic pain, and poststroke pain.

According to particularly preferred embodiments of the invention, the patient
suffers from ongoing symptomatic COVID-19 (NICE guideline [NG188]);
prior to infection with the coronavirus, neither suffered from (i) pre-existing neurological injury nor from (ii) pre-existing neuropathic pain;
   wherein the pain to be treated is neuropathic pain, preferably peripheral neuropathic pain; more preferably COVID-19 neuropathic pain.

According to particularly preferred embodiments of the invention, the patient
suffers from post-COVID-19 syndrome (NICE guideline [NG188]);
prior to infection with the coronavirus, suffered from (i) pre-existing neurological injury and/or (ii) pre-existing neuropathic pain, preferably pre-existing chronic neuropathic pain, more preferably preexisting chronic polyneuropathic pain or pre-existing chronic mononeuropathic pain;
   wherein the pain to be treated is neuropathic pain, preferably peripheral neuropathic pain; more preferably selected from the group consisting of COVID-19 neuropathic pain, postherpetic neuralgia (PHN), chronic painful radiculopathy, cervical radiculopathy, diabetic painful neuropath (DPN), spinal cord injury pain, neuropathic pain due to myelitis, neuropathic pain due to cancer, trigeminal neuralgia, neuropathic pain associated with Guillain-Barré syndrome, chemotherapy induced neuropathic pain, small-fiber neuropathy, chronic idiopathic axonal polyneuropathy, post-traumatic neuropathic pain, post-surgical neuropathic pain, HIV infection-induced neuropathic pain, and poststroke pain.

According to particularly preferred embodiments of the invention, the patient
suffers from post-COVID-19 syndrome (NICE guideline [NG188]);
prior to infection with the coronavirus, has suffered from (i) pre-existing neurological injury and/or (ii) pre-existing neuropathic pain, preferably pre-existing chronic neuropathic pain, more preferably pre-existing chronic polyneuropathic pain or pre-existing chronic mononeuropathic pain;
   wherein the pain to be treated is neuropathic pain, preferably peripheral neuropathic pain; more preferably selected from the group consisting of COVID-19 neuropathic pain, postherpetic neuralgia (PHN), chronic painful radiculopathy, cervical radiculopathy, diabetic painful neuropath (DPN), spinal cord injury pain, neuropathic pain due to myelitis, neuropathic pain due to cancer, trigeminal neuralgia, neuropathic pain associated with Guillain-Barré syndrome, chemotherapy induced neuropathic pain, small-fiber neuropathy, chronic idiopathic axonal polyneuropathy, post-traumatic neuropathic pain, post-surgical neuropathic pain, HIV infection-induced neuropathic pain, and poststroke pain.

According to particularly preferred embodiments of the invention, the patient
suffers from post-COVID-19 syndrome (NICE guideline [NG188]);
prior to infection with the coronavirus, neither suffered from (i) pre-existing neurological injury nor from (ii) pre-existing neuropathic pain;
   wherein the pain to be treated is neuropathic pain, preferably peripheral neuropathic pain; more preferably COVID-19 neuropathic pain.

According to particularly preferred embodiments of the invention, the patient
suffers from long COVID (NICE guideline [NG188]);
prior to infection with the coronavirus, suffered from (i) pre-existing neurological injury and/or (ii) pre-existing neuropathic pain, preferably pre-existing chronic neuropathic pain, more preferably preexisting chronic polyneuropathic pain or pre-existing chronic mononeuropathic pain;
   wherein the pain to be treated is neuropathic pain, preferably peripheral neuropathic pain; more preferably selected from the group consisting of COVID-19 neuropathic pain, postherpetic neuralgia (PHN), chronic painful radiculopathy, cervical radiculopathy, diabetic painful neuropath (DPN), spinal cord injury pain, neuropathic pain due to myelitis, neuropathic pain due to cancer, trigeminal neuralgia, neuropathic pain associated with Guillain-Barre syndrome, chemotherapy induced neuropathic pain, small-fiber neuropathy, chronic idiopathic axonal polyneuropathy, post-traumatic neuropathic pain, post-surgical neuropathic pain, HIV infection-induced neuropathic pain, and poststroke pain.

According to particularly preferred embodiments of the invention, the patient
suffers from long COVID (NICE guideline [NG188]);
prior to infection with the coronavirus, has suffered from (i) pre-existing neurological injury and/or (ii) pre-existing neuropathic pain, preferably pre-existing chronic neuropathic pain, more preferably pre-existing chronic polyneuropathic pain or pre-existing chronic mononeuropathic pain;
   wherein the pain to be treated is neuropathic pain, preferably peripheral neuropathic pain; more preferably selected from the group consisting of COVID-19 neuropathic pain, postherpetic neuralgia (PHN), chronic painful radiculopathy, cervical radiculopathy, diabetic painful neuropath (DPN), spinal cord injury pain, neuropathic pain due to myelitis, neuropathic pain due to cancer, trigeminal neuralgia, neuropathic pain associated with Guillain-Barré syndrome, chemotherapy induced neuropathic pain, small-fiber neuropathy, chronic idiopathic axonal polyneuropathy, post-traumatic neuropathic pain, post-surgical neuropathic pain, HIV infection-induced neuropathic pain, and poststroke pain.

According to particularly preferred embodiments of the invention, the patient
suffers from long COVID (NICE guideline [NG188]);
prior to infection with the coronavirus, neither suffered from (i) pre-existing neurological injury nor from (ii) pre-existing neuropathic pain;
   wherein the pain to be treated is neuropathic pain, preferably peripheral neuropathic pain; more preferably COVID-19 neuropathic pain.

In preferred embodiments, Tapentadol is administered
(i) orally; and/or
(ii) twice daily; and/or
(iii) in form of a dosage form providing prolonged release.

Preferably, Tapentadol is administered at a dose of 25 mg, 50 mg, 100 mg, 150 mg, 200 mg, or 250 mg, in each case expressed as dose equivalent relative to the non-salt form of Tapentadol.

The patients to be treated in accordance with the invention are not particularly limited. In preferred embodiments, the patients are female. In other preferred embodiments, the patients are male. In preferred embodiments, the patients are adults. In preferred embodiments, the patients are elderly, preferably are at least 60 years, more preferably at least 65 years.

In preferred embodiments, especially when the acute phase of COVID-19 is still ongoing, the patient receives additional medicaments for treating viral infection, signs and/or symptoms thereof. Such additional medicaments are not particularly limited and may contain as pharmacologically active ingredients e.g. Dexamethasone, Remdesivir, Chloroquine, Lopinavir, or Ritonavir.

According to S. Natoli et al., Eur Rev Med Pharmacol Sci, 2021, 25: 4854-4867, which was published after the priority date (May 7, 2021), Tapentadol is associated with a low risk of drug-drug interactions at the CYP450 level and may, therefore, be used in poly-treated patients. Furthermore, Tapentadol exhibits a lower immunosuppressive activity at analgesic doses vs. Morphine. The major elimination pathway for Tapentadol is conjugation with glucuronic acid mediated via UGT mainly UGT1A6, UGT1A9 and UGT2B7 isoforms. Thus, concomitant administration with strong inhibitors of these isoenzymes may lead to increased systemic exposure of Tapentadol. Tapentadol is preferred over opioids metabolized through the CYP450 hepatic system.

The following example further illustrates the invention but is not to be construed as limiting its scope.

### EXAMPLE 1

The 16 patients as compiled in the following table receive Tapentadol orally twice daily at dosages within the range of from 50 mg to 250 mg (Palexia® retard) for treating neuropathic pain:

| | 1 | 2 | 3 | 4 | 5 | 6 | 7 | 8 | 9 | 10 | 11 | 12 | 13 | 14 | 15 | 16 |
|---|---|---|---|---|---|---|---|---|---|---|---|---|---|---|---|---|
| age | 67 | 66 | 53 | 45 | 23 | 22 | 53 | 48 | 35 | 93 | 37 | 43 | 42 | 50 | 83 | 18 |
| gender | F | M | M | M | F | F | M | M | M | F | M | F | F | M | F | F |
| COVID status | A | A | A | A | O | O | O | O | P | P | P | P | L | L | L | L |
| COVID symptoms | − | + | − | + | − | + | − | + | − | + | − | + | − | + | − | + |
| during acute phase | | | | | | | | | | | | | | | | |
| pre-existing neuro- | — | — | 11 | 10 | — | — | 8 | 7 | — | — | 4 | 4 | — | — | 1 | 1 |
| pathic pain | | | | | | | | | | | | | | | | |
| pain treated | 12 | 12 | 11 | 10 | 9 | 9 | 8 | 7 | 6 | 5 | 4 | 4 | 3 | 2 | 1 | 1 |

F = female;
M = male;
A = acute COVID-19;
O = ongoing symptomatic COVID-19;
P = post-COVID-19 syndrome;
L = long COVID.
pre-existing neuropathic pain/pain treated:
1 postherpetic neuralgia PHN
2 chronic painful radiculopathy
3 cervical radiculopathy
4 diabetic painful neuropath DPN
5 spinal cord injury pain
6 neuropathic pain due to myelitis
7 neuropathic pain due to cancer
8 trigeminal neuralgia
9 neuropathic pain associated with Guillain-Barre syndrome
10 chemotherapy induced neuropathic pain
11 HIV infection-induced neuropathic pain
12 poststroke neuropathic pain

1.-40. (canceled)

41. A method of treating pain in a patient who is infected with a coronavirus and suffers from a corona virus disease (COVID), wherein the pain to be treated is neuropathic pain due to infection with the coronavirus, said method comprising administering to said patient an effective amount therefor of Tapentadol or a physiologically acceptable salt thereof.

42. The method according to claim 41, wherein the patient suffers from COVID-19.

43. The method according to claim 41, wherein the pain to be treated is peripheral neuropathic pain.

44. The method according to claim 41, wherein the pain to be treated is chronic.

45. The method according to claim 41, wherein the patient receives an additional medicament for treating viral infection, signs and/or symptoms thereof.

46. The method according to claim 45, wherein the patient receives an additional medicament for treating corona virus infection, signs and/or symptoms thereof.

47. The method according to claim 45, wherein said additional medicament contains as pharmacologically active ingredient Dexamethasone, Remdesivir, Chloroquine, Lopinavir, or Ritonavir.

48. A method of treating neuropathic pain in a patient
who is infected with a severe acute respiratory syndrome-related coronavirus and suffers from a corona virus disease (COVID), and
who receives an additional medicament for treating viral infection, signs and/or symptoms thereof,
said method comprising administering to said patient an effective amount therefor of Tapentadol or a physiologically acceptable salt thereof.

49. The method according to claim 48, wherein the additional medicament is for treating corona virus infection, signs and/or symptoms thereof.

50. The method according to claim 48, wherein the coronavirus is a severe acute respiratory syndrome coronavirus 42.

51. The method according to claim 48, wherein the patient suffers from COVID-19.

52. The method according to claim 51, wherein the acute phase of COVID-19 is still ongoing.

53. The method according to claim 48, wherein said additional medicament contains as pharmacologically active ingredient Dexamethasone.

54. The method according to claim 48, wherein said additional medicament contains as pharmacologically active ingredient Remdesivir.

55. The method according to claim 48, wherein said additional medicament contains as pharmacologically active ingredient Chloroquine.

56. The method according to claim 48, wherein said additional medicament contains as pharmacologically active ingredient Lopinavir.

57. The method according to claim 48, wherein said additional medicament contains as pharmacologically active ingredient Ritonavir.
